(19) United States
(12) Patent Application Publication
(10) Pub. No.: US 2022/0387550 A1
(43) Pub. Date: Dec. 8, 2022

(54) LANTIBIOTIC SOLUTION AGAINST BACTERIAL INFECTIONS

(71) Applicant: STERIFY S.R.L., Torino (TO) (IT)

(72) Inventors: Mauro FIORINI, Granarolo Dell'Emilia (BO) (IT); Paolo FATTORI, Borgo Maggiore (SM) (SM)

(21) Appl. No.: 17/775,983

(22) PCT Filed: Nov. 19, 2020
(86) PCT No.: PCT/IB2020/060902

§ 371 Date: May 11, 2022

(30) Foreign Application Priority Data
Nov. 22, 2019 (IT) 102019000021882

(51) Int. Cl. A61K 38/16; A61K 9/08; A61K 47/10; A61K 47/32; A61K 47/12

(52) U.S. Cl. CPC A61K 38/164; A61K 9/08; A61K 47/10; A61K 47/32; A61K 47/12

(57) ABSTRACT

A lantibiotic solution against bacterial infections comprises an aqueous solvent, a water-soluble polymeric component, class I bacteriocins, and amphipathic molecules.

### TECHNICAL FIELD

This invention relates to a lantibiotic solution against bacterial infections.

The problem of bacterial resistance to antibiotics is increasing and considerable, in particular for some forms of infection. Well thought-out use of antibiotic therapy, with a targeted selection of molecules, is a fundamental requirement for the success of the individual treatment and for the effectiveness of future antibacterial therapies.

In Italy, approximately 8% of hospitalised patients contract an infection associated with treatment procedures and, of these, 20-22% are infections of the surgical site. In the United States of America each year 300,000 cases of post-surgical infection are recorded. Specifically, approximately 1 infection in 100 is derived from prosthetic surgical activity.

Joint infections may occur in the wound or deep down around artificial implants. An infection may develop during the stay in hospital or after returning home. Joint replacement infections may even appear years after the operation.

At the time of the surgery various measures are adopted for minimising the risk of infection but, to this day, the most important measures known for reducing the risk of infection after prosthetic surgical operations still include only rather dated strategies.

Indeed it is customary to prescribe antibiotics before and after the surgical operation, which are in general administered from one hour before the operation (usually already in the operating theatre) and, at intervals, up to 24 hours after the operation.

Efforts are also made to minimise the length of the operation, so as to reduce the risk by limiting the open wound exposure time, and to limit the number of people entering and exiting the operating theatre. Particular attention is paid to the sterility of the operating site and to sterilization of the surgical instruments in an autoclave, as well as to the correct packaging of implants in a sterile environment, for ensuring the absence of any type of contamination.

Finally, a long-term preventive treatment is often prescribed, again antibiotic-based; it is very widespread in the execution of dental prosthetic operations, the aim being to protect the implants from bacteria, but there is a lack of objective evidence of its effectiveness.

### BACKGROUND ART

Certainly, frequent (and often unnecessary) use of antibiotics has promoted the growing resistance of pathogenic agents; on the other hand, a reduction in the discovery of new and effective antibiotics has promoted the development of alternative approaches in antimicrobial therapy.

These include the use of bacteriocins. Unlike antibiotics, which have a broad spectrum of activity, bacteriocins are usually active against particular types of bacteria, closely linked to the strain from which they have been produced.

Moreover, they are active in nanomolar concentrations, whilst antibiotics are effective at much higher concentrations.

In recent years attention has focused on bacteriocins from gram-positive bacteria, in particular lactic bacteria: the status of “generally considered as safe” of these organisms, their application in the food sector and as probiotics has led to an increase in research activity even on the antimicrobial peptides that they produce.

Some bacteriocins are effective against multi-drug resistant pathogens, such as against some strains of Staphylococcus Aureus which have become resistant to methicillin and other beta-lactam antibiotics, or against vancomycin-resistant enterococci.

Bacteriocins are grouped in three main classes: class I bacteriocins are called lantibiotics and are split into two sub-groups, based on the structure and the mechanism of action; class II bacteriocins are heat-stable, formed by peptides which do not contain lanthionine and are split into four sub-groups; class III bacteriocins comprise bacteriolysins.

In particular, lantibiotics act on the bacterial membrane, forming pores through which cytoplasmic material comes out or blocking cellular metabolism thanks to an interaction with the bacterial enzymes.

However, unfortunately the spectrum of activity of lantibiotics is substantially limited to Gram positive bacteria, which have a cell wall that is easier to attack, whose outermost layer is constituted of peptidoglycan, whilst Gram negative bacteria have an outer membrane, made up of phospholipids and lipopolysaccharides, on which the pore-forming action of lantibiotics is not effective.

Although in the food industry some time ago solutions were devised to give bacteriocins a broad spectrum of action, they cannot be used in the medical/surgical sphere: indeed, they involve the use of chelating agents or essential oils, or sanitization treatments with sodium hypochlorite or surfactants such as Tween 80 (as suggested by document CN 109 588 613), which do not promote, or which even hamper, the regeneration of tissues.

It has also been shown that topical use of Nisin could significantly speed up the healing process of burn wounds infected with Staphylococcus aureus, a Gram positive bacterium, but has no effect on burn wounds infected with Escherichia coli, a Gram negative bacterium, as can be inferred from the publication in Biomedical Materials of the article “Precise management of chronic wound by nisin with antibacterial selectivity” (7 May 2019).

Moreover, patent document WO 2004/052308 discloses a method for topical treatment of infections based on a peptidase of bacterial origin combined with Nisin and carried by an emulsion containing up to 10% of surfactants (SEPIGEL 305 or SIMUGEL 600), which cannot be used in the surgical sphere.

### Disclosure of the Invention

The aim of this invention is therefore to eliminate the above-mentioned disadvantages.

The invention, with the features described in the claims, achieves the aim using hydroxytyrosol combined with bacteriocins.

The main advantage obtained by means of this invention is essentially the fact that it is effective both against Gram positive bacteria and Gram negative bacteria, aiding the regeneration of contaminated connective tissues.

Moreover, used as a treatment, the invention prevents, or at least hampers, colonization by the bacteria responsible for infections at surgical implant sites.

Further advantages and features of the invention will be more apparent in the detailed description below.

### PREFERRED EMBODIMENTS OF THE INVENTION

A lantibiotic solution against bacterial infections comprises an aqueous solvent, a water-soluble polymeric component, class I bacteriocins, and hydroxytyrosol. The mixing in millimolar concentrations of the bacteriocins with hydroxytyrosol allows the prevention and treatment of bacterial infections and/or prevention of the formation of biofilms at surgical operation sites (for example, in the implant of prosthetic devices) and in infected periodontal or peri-implant pockets; and also speeds up healing of the extracellular matrix of the tissues. The class I bacteriocins, also called lantibiotics because they contain modified amino acids of the lanthionine type, are capable, aided by the antioxidant and solubilizing action of the hydroxytyrosol, both of forming pores on the membranes of the bacteria, both Gram positive and Gram negative, causing their cell death and thereby eradicating the cause of possible infections, and, at the same time, of activating the cells of the surrounding tissue to rebuild the damaged extracellular matrix.

In the aqueous solution, the concentration of the polymeric component is between 1 and 30% by weight. The concentration of the class I bacteriocins is between 1 and 10 mM, whilst the concentration of hydroxytyrosol is less than or equal to 100 mM.

The class I bacteriocins are selected from a group comprising Nisin Z, Nisin A and/or Nisin F, whilst the water-soluble polymeric component is preferably selected from a group comprising a polymer and/or polymeric mixtures and/or copolymers of ethylene oxide (PEG, PEO, POE), vinyl alcohol, vinyl pyrrolidone, lactic acid.

1. A lantibiotic solution against bacterial infections, wherein it comprises an aqueous solvent, a water-soluble polymeric component, class I bacteriocins, and hydroxytyrosol.

2. The lantibiotic solution according to claim 1, wherein the concentration of the polymeric component is between 1 and 30% by weight.

3. The solution according to claim 1, wherein the concentration of the class I bacteriocins is between 1 and 10 mM.

4. The solution according to claim 1, wherein the concentration of hydroxytyrosol is less than or equal to 100 mM.

5. The solution according to claim 1, wherein the class I bacteriocins are selected from a group comprising Nisin Z, Nisin A and/or Nisin F.

6. The solution according to claim 1, wherein the water-soluble polymeric component is selected from a group comprising a polymer and/or polymeric mixtures and/or copolymers of ethylene oxide (PEG, PEO, POE), vinyl alcohol, vinyl pyrrolidone, lactic acid.
